(19) United States
(12) Plant Patent Application Publication
(10) Pub. No.: US 2004/0019940 P1
(43) Pub. Date: Jan. 29, 2004

(54) Hybrid carnation plant named ' CFPC SARI '

(76) Inventor: Oscar T. Hasegawa, Salinas, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/208,168

(22) Filed: Jul. 29, 2002

(51) Int. Cl. A01H 5/00

(52) U.S. Cl. PLT/273

(57) ABSTRACT

A new variety of carnation plant producing attractive yellow picotee purple flowers.

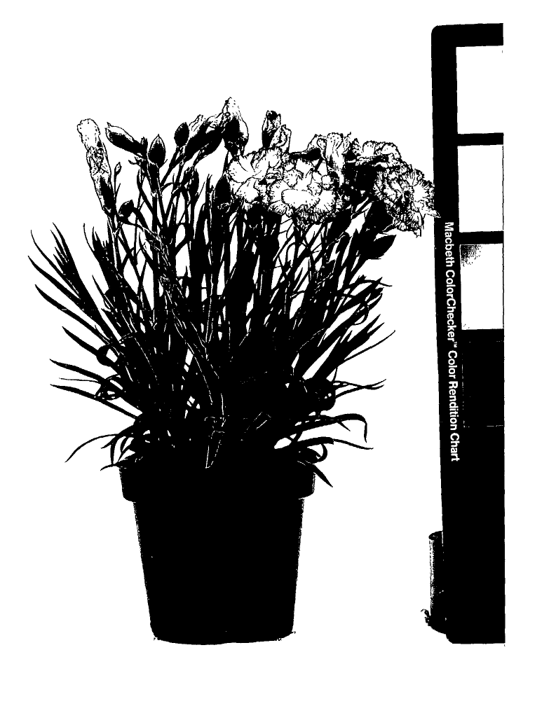

### BACKGROUND OF THE INVENTION

The present invention relates to a new and distinct variety of carnation plant which was originated by me by crossing two undistributed unpatented seedlings identified as ‘970135’ (seed parent) and ‘970111’ (pollen parent). The varietal denomination of the new variety is ‘CFPC SARI’.

### SUMMARY OF THE INVENTION

Among the novel characteristics possessed by the new variety which distinguish it from other varieties of which of I am aware, are its attractive yellow picotee purple flowers which retain color without fading. When grown in a greenhouse in Salinas, Calif., in 4½ inch plastic pots, plants are about 8½ inches tall and about 5 inches in diameter. Flower diameter averages about 2 inches with a crown of about ¾ inch (average). Foliage is about 3 inches long and about ¼ inch wide at its widest point.

Asexual reproduction by propagation by cuttings of the new variety as performed in Salinas, Calif., shows that the foregoing and other distinguishing characteristics come true to form and are established and transmitted through succeeding propagation.

### BRIEF DESCRIPTION OF ILLUSTRATION

The accompanying illustration shows typical specimens of the vegetative growth and flowers of the new variety depicted in color as nearly true as it is reasonably possible to make the same in color illustration of this character.

### DESCRIPTION OF THE NEW VARIETY

The following is a detailed description of my new variety, with color terminology in accordance with The Royal Horticultural Society Colour Chart (RHSCC). The terminology used in color description herein refers to plate numbers in the aforementioned color chart. Phenotypical expression may vary depending upon varying environmental, seasonal and cultural conditions.

The information provided herein is based on observations of plants grown inside commercial greenhouses at Salinas, Calif. The height of plant and flower and foliage size are established as an average using standard commercial cultural practices.

Classification:

Botanical.—Hybrid Dianthus caryophyllus.

Commercial.—Carnation, standard disbud, greenhouse type, suitable for cut flower production and as a pot spray.

Plant:

Height.—About 8½ inches.

Form.—Herbaceous.

Growing habit.—Semi-upright.

Blooming habit.—As a spray (pot).

Blooming season.—Year round.

Rooting time/habit.—About 18 days.

Breaking action.—Good.

Mutation prone.—No.

Disease resistance.—Good, resistence to soil-borne diseases such as Fusarium oxysporum, and F. graminearum and root-rots such as damping off, water molds and the like as is typical of carnations.

Temperature tolerance.—Wide range.

Foliage (Base of plant):

Color.—Upper side: Near 147A. Lower side: Near 137A.

Size.—About 3 inches long and about ¼ inch wide.

Quantity.—Numerous.

Shape.—Lanceolate.

Texture.—Smooth.

Ribs.—Central keel.

Arrangement.—Bi-lateral along stem.

Stem.—Color: Near 139A.

Bud.—Shape: Ovoid. Color: Near 143C. Length: About 1 1/16 inches. Diameter: About ⅜ inch.

Calyx.—Sepals: About 5. Length: About 5/16 inch. Width: About ¼ inch. Color. Outside: Near 144A. Inside: Near 147D.

Bloom:

Shape.—Rounded.

Size.—Diameter: About 2 inches. Depth: About ¾ inch.

Borne.—As a spray.

Color.—General tonality: Yellow and purple. Upper side: Near 4D. Lower side: Near 4D. Center: Near 4D. Outer petals: Near 80A.

Permanence.—Dry up on receptacle.

Keeping quality.—Very good.

Fragrance.—Strong perfume.

Petalage:

Quantity.—About 24.

Outer petal size.—Length: About 1¾ inches. Width: About 1⅛ inches.

Central petal size.—Length: About 1⅝ inches. Width: About 15/16 inch.

Texture.—Smooth.

Appearance.—Shiny.

Form.—Fan shaped.

Reproductive organs:

Stamen.—None.

Anthers.—None.

Filaments.—None.

Pistil.—1. Length: About 1⅛ inches. Color: Whiter than 155D.

Stigma.—2. Color: Near 4D.

Fertility.—Male: Sterile. Female: Good.

Ovary.—Shape: Conical. Color. Base: Near 155D. Body: Near 144C. Apex: Near 144C.

I claim:

1. A new and distinct carnation plant of the variety substantially as herein shown and described.
